(19) United States
(12) Patent Application Publication
(10) Pub. No.: US 2006/0178705 A1
(43) Pub. Date: Aug. 10, 2006

(54) Heart rate variance cardiac pacemaker

(76) Inventor: Paul Janssen, Dublin, OH (US)

Correspondence Address: ROETZEL & ANDRESS, 1375 EAST 9TH STREET, CLEVELAND, OH, 44114, US

(21) Appl. No.: 11/351,729

(22) Filed: Feb. 10, 2006

Related U.S. Application Data
(60) Provisional application No. 60/652,391, filed on Feb. 10, 2005.

(51) Int. Cl. A61N 1/362

(52) U.S. Cl. 607/9

(57) ABSTRACT

Various embodiments of a cardiac pacemaker device and methods are provided. In one embodiment a cardiac pacemaker device includes a pulse generator which emits a plurality of stimulation pulses such that each of the plurality of stimulation pulses is separated by a plurality of inter-beat durations, respectively. The pacemaker device further includes a pacing algorithm configured to implement the pulse generator to generate alternately shorter and longer inter-beat durations independent of hemodynamic loading conditions and such that the average inter-beat duration remains constant.

### RELATED APPLICATION

This application is a conversion of U.S. Provisional Patent Application No. 60/652,391, filed Feb. 10, 2005.

### FIELD OF THE INVENTION

The present invention is in the general field of medicine and, more particularly, in the field of medical devices and electronic medical devices and treatments.

### BACKGROUND OF THE INVENTION

Heart rate variability (HRV), or the changes in inter-beat durations of the heart, occurs naturally and is influenced by several factors, including baroreceptor reflexes and respiratory activity. A decrease in HRV is known to occur with aging as well as in several of the most common cardiovascular disorders that affect modern society, and is altered under various pathological conditions. Clinical conditions such as congestive heart failure, diabetic neuropathy and ischemic heart disease all lead to a decrease in HRV. Until now, HRV has been exclusively acknowledged only as an indicator both of the autonomic status of the organism as well as a marker of some prognostic significance in both diseased and non-diseased states. It is however incompletely understood if and/or how HRV affects myocardial contractility, how increased inter-beat periods lead to increase loading of the heart, and how the resulting strength of the heartbeat is affected by a combination of contractility and loading.

HRV is both a dynamic indicator of physiologic states of the organism (active stress) as well as a marker of the cumulative load on the heart (e.g. decrease in HRV observed with aging). HRV is subject to multiple influences derived from the autonomic system (baro-receptor reflex, calcium channel blockers, beta blockers, etc.) as well as those intrinsic to the firing of the sino-atrial node affect HRV. Clinical conditions such as congestive heart failure (CHF) diabetic neuropathy and ischemic heart disease all lead to a decrease in HRV. Heart rate variability has also been reported to be predictive not only of increased mortality in diseased states buy also as an indicator of morbidity in healthy individuals. More recently, analysis of HRV has been increasingly used in order to provide predictive information relative to morbidity and mortality in patients undergoing pre-operative evaluation. While the measurements of HRV have always been seen as reflecting the balance (or imbalance) of the components of the autonomic system, it would be desirable to know whether the change in HRV has an effect on myocardial contractility.

### SUMMARY OF THE INVENTION

The present invention involves a method of introducing HRV as a therapeutic tool that can have both immediate direct and long-term indirect effects on cardiac contraction strength. It has been found that HRV is a contributor to contractility.

In one embodiment the present invention provides for a method that includes inducing a plurality of stimulation pulses to the heart, each of which is separated by a plurality of inter-beat durations, respectively; and generating alternately shorter and longer inter-beat durations independent of hemodynamic loading conditions while the average inter-beat duration remains constant.

In another embodiment, the present invention provides for a cardiac pacemaker device which includes a pulse generator that emits a plurality of stimulation pulses each of which is separated by a plurality of inter-beat durations, respectively. The pacemaker device also includes a pacing algorithm configured to implement the pulse generator to generate alternately shorter and longer inter-beat durations independent of hemodynamic loading conditions and such that the average inter-beat duration remains constant.

It has been found that HRV can strengthen the average contractile force of the heart, without increasing heart rate, and improves protein expression patterns in the heart, thereby further improving cardiac function and efficiency. Such a variable rate pacemaker device is therefore superior to a conventional, fixed rate, pacemaker, and is beneficial to those who use a pacemaker, and also to those with weak or weakened contractile force, i.e., a large percentage of all cardiac patients, and who would not typically use a pacemaker.

### BRIEF DESCRIPTION OF THE FIGURES

FIG. 1 shows a curve of contractile force versus various magnitudes of variation in inter-beat duration based on an average of 4 Hz frequency, according to an embodiment of the invention;

FIG. 2 shows the contractile force versus frequency at 40% variability of inter-beat duration, according to an embodiment of the invention;

FIG. 3 shows a plot of the force of contraction versus inter-beat duration during variable pacing superimposed upon a baseline cycle time of 125 milliseconds;

FIG. 4 shows the tracings of intracellular calcium, concentration, stimulation pulse, and force of contraction over a period of several seconds, according to an embodiment of the invention; and

FIG. 5 shows a plot of the amplitude of the calcium signal, versus amplitude of the force signal at variable inter-beat duration, according to an embodiment of the invention.

### DESCRIPTION

It has been found that HRV can be a modulator of contractility, and this modulating capacity can be used therapeutically. Re-introduction of HRV via a novel pacemaking algorithm can be used to introduce a positive and immediate effect on contraction, as well as a longer-term effect though improved protein expression in the heart, as will be further described.

In one embodiment the present invention provides for a method that includes inducing a plurality of stimulation pulses to the heart, each of which is separated by a plurality of inter-beat durations, respectively; and generating alternately shorter and longer inter-beat durations independent of hemodynamic loading conditions while the average inter-beat duration remains constant. The variation in inter-beat durations can range from about 1% to about 100%, in an alternative embodiment from about 5% to about 100%, and in yet another embodiment from about 10% to about 20%, based on the average inter-beat duration of the heart when no stimulation pulses are induced. In addition, the alternately shorter and longer inter-beat durations can be generated on a randomized basis. The stimulation pulses are used to increase the intracellular calcium handling within the heart.

In another example embodiment, the method includes inducing a plurality of stimulation pulses such that they alternate between fixed steady-state frequency and variable frequencies, while the average inter-beat duration remains constant.

A protocol that distinguished loading effects from contractility effects was employed and experiments were conducted using cardiac trabeculae dissected from the right ventricle of rat hearts, as described in the examples below.

At other rates of variability, ranging from 10-120%, no variation was observed between fixed and variable pacing. However, a positive and very tight correlation was observed between the inter-beat duration and strength of the following beat (0.103±0.016 ms-1, P<0.05, n=10). At 6 Hz and 8 Hz pacing rate (which is physiological heart rate for the rat), we did observe a positive effect of variable pacing. When paced with 40% variability, both at 6 Hz and 8 Hz, average contractile force was higher when compared to fixed-rate pacing.

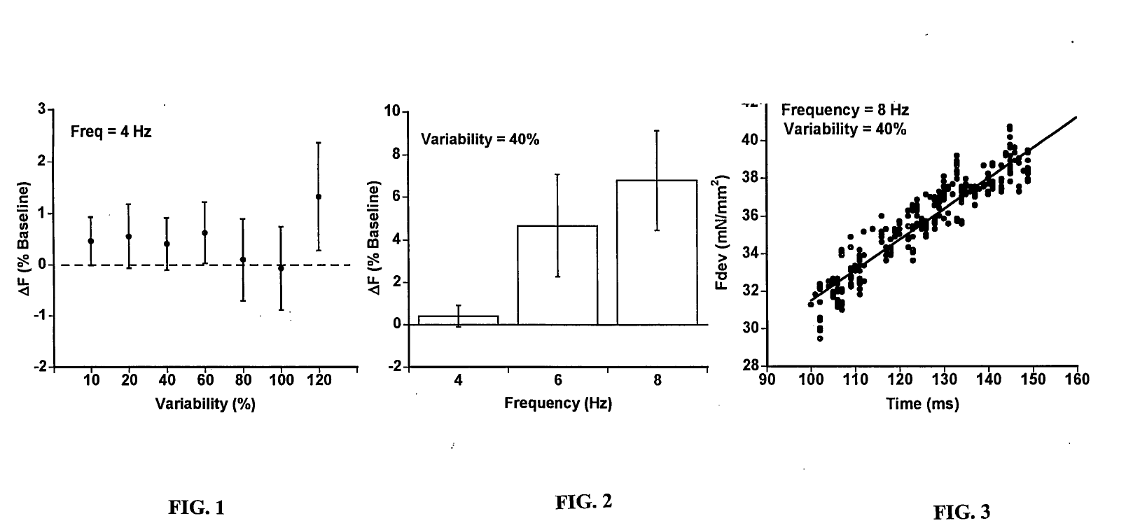

FIG. 1 shows that at 4 Hz, no loss of contractile force is observed, and an increase is observed with 120% variability of the inter-beat duration. FIG. 2 shows that at a 40% inter-beat duration variability, no increase is observed at a baseline inter-beat duration of 250 ms, but that a 40% variability at an inter-beat duration of 166 ms and 125 ms, the average contractile force is higher compared to a fixed inter-beat duration. FIG. 3 shows that at a baseline inter-beat duration of 125 ms with a 40% variability, the force of contraction is closely correlated with inter-beat duration. At fixed rate inter-beat duration, force of this muscle was 32.1 mN/mm2, below the average of the variable duration (which was 35.3 mN/mm2 in this example).

At the conditions as outlined HRV can change the beat-to-beat contractile strength independent of loading conditions, and average force production can be increased under certain conditions by introducing the variable pacing component. The application of HRV at other values and ranges of these and other variables may also have positive effects on contractile strength. Under no condition tested we observed negative effect on average force development.

The immediate or short-term effect is that the application of a random portion to inter-beat timing will not change the average number of beats per time unit (basically; heart rate), but does impact positively on the contractile strength on average. At a rate of 8 Hz, which is very much physiological for the rat, force of contraction was increased with random pacing versus standard steady-state pacing. A positive tight correlation between inter-beat duration and strength of the following beat (0.103±0.0116 ms-1) was observed suggesting that HRV does change beat to beat contractile strength, independent of loading conditions, for example at a resting condition without exercise.

In addition, HRV pacing can introduce additional benefits in the long-term. It has been observed herein that the amplitude of the calcium transient, for example the intracellular calcium handling within the heart, depends on the inter-beat duration. That is, a variance in heart rate will directly lead to a larger variance in intracellular calcium transients compared to the standard, or steady-state, pacing. This complacency can result in improved intracellular signaling, possibly combating or reversing remodeling that has occurred due to various pathogenesis. Although not wishing to be bound by a particular theory, it is believed that reintroduction of variability may thus initiate establishment of a healthier protein expression pattern, with the result of improved contractility of the heart.

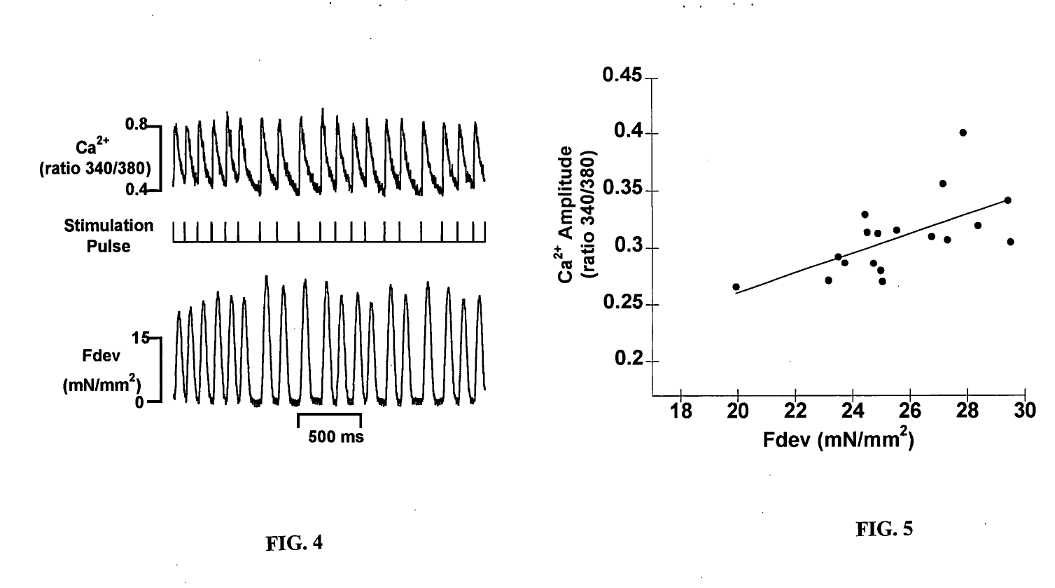

It has also been found that a correlation exists between the force development of the myocardium and the amplitude of the calcium transient. As a result of HRV pacing, the average intracellular calcium transient can increase in amplitude, which leads to improved calcium handling. As heart failure and ageing introduce impaired calcium handling, improvement of calcium handling in the long run should benefit the heart by improving ion homeostasis. FIG. 4 shows three tracings over time, the intracellular Calcium (top), the stimulation pulse (middle), and the force of contraction. Over a period of several seconds, the force and calcium were measured simultaneously. FIG. 5 shows a positive correlation exists between amplitude of the calcium signal, and amplitude of the force signal that the variations in force that arise at variable inter-beat duration are the result of altered calcium transient amplitude, which provides an explanation for the mechanism of the findings.

In additional experiments, the basic protocol of HRV on isometrically contracting trabeculae has been executed with canine myocardium. The canine mammal exhibits closer resemblance to the human in vivo situation, and the results obtained with canine myocardium are very similar to those observed in rats. In canine myocardium, a strong positive correlation exists between the duration of the previous beat and contractile strength. Although assessed at lower frequencies to reflect the natural range of the dog (e.g. 1-4 Hz), we observed a small increase in contractile strength under certain conditions, while no loss of contractile strength was found in any other tested condition. The results of the canine experiments indicate that the phenomenon of positive HRV effects is present in larger mammals.

In another embodiment, the present invention provides for a cardiac pacemaker device that includes a pulse generator which emits a plurality of stimulation pulses, each of which is separated by a plurality of inter-beat durations, respectively; and a pacing algorithm configured to implement the pulse generator to generate alternately shorter and longer inter-beat durations independent of loading conditions and such that the average inter-beat duration remains constant. The inter-beat durations can range from about 1% to about 100%, in an alternative embodiment from about 5% to about 100%, and in yet another embodiment from about 10% to about 20%, and all ranges therebetween based on the average inter-beat duration of the heart when no stimulation pulses are induced. The cardiac pacemaker device can induce pulses that alternately shorter and longer inter-beat durations are generated on a randomized basis. Optionally, the stimulation pulses are induced at a pattern that increases the average contractile strength of the heart without increasing average inter-beat duration. The randomized stimulation pulses of the cardiac pacemaker device can be induced at a constant average frequency that is less than about 4 Hz, in an alternative embodiment at a frequency that ranges from about 0.5 Hz to about 3 Hz, and in yet another embodiment, at a frequency that ranges from about 1 Hz to about 2 Hz, and all ranges therebetween. In addition, the randomized stimulation pulses can be induced by independent changes in the magnitude of variation and the duration of the average inter-beat variation. In another embodiment, the cardiac pacemaker device can generate stimulation pulses that alternate between fixed steady state frequency and variable frequencies, while the average inter-beat duration remains constant.

The cardiac pacemaker device of the various example embodiments described above can further include a pulse actuator that has a first state and a second state. The pacing algorithm, which can be embedded on a microprocessor, for example, can be configured to implement the pulse generator such that each of the plurality of stimulation pulses is separated by an inter-beat duration which is constant when the pulse actuator is in the first state. When the pulse actuator is in the second state, the pacing algorithm is configured to implement the pulse generator to alternately shorten and lengthen the inter-beat duration, while maintaining the average inter-beat duration.

### EXAMPLES

Small isolated cardiac trabeculae (˜150 μm diameter, length ˜2-3 mm), were carefully dissected from the right ventricle from rat hearts. At 37° Celsius, these trabeculae were stimulated isometrically at a fixed rate and at intervals with a variable component that ranged from about 10-120% variability baded on the average inter-beat duration of the heart. Variability was distributed linearly, and expressed as a percentage of the variation between the longest and shortest beat compared to steady state (e.g. at 40% variation at 4 Hz, inter-beat duration varied from 200 to 300 ms). The mean average developed for a 5 minute period during variable pacing was analyzed, and compared to the steady state response at the base frequency. The number of beats during the 5 minute period was identical between variable and steady pacing. No significant changes in mean average force were observed between fixed (28.1±5.8 mN/mm2) and 40% variable pacing (28.3±5.7 mN/mm2) at 4 Hz. Data from the experiments are illustrated in FIG. 1-3 as described above.

It will be appreciated that modifications to the method may be suggested by those skilled in the art, and that the cardiac pacemaker device may adopt a wide variety of configurations. It should be understood that the present invention is intended to include such modifications and alterations in so far as they fall within the scope of the appended claims or the equivalents thereof.

What is claimed is:

1. A method comprising:
inducing a plurality of stimulation pulses to the heart such that each of the plurality of stimulation pulses is separated by a plurality of inter-beat durations, respectively; and
generating alternately shorter and longer inter-beat durations independent of hemodynamic loading conditions and such that the average inter-beat duration remains constant.

2. The method of claim 1, wherein the variation in inter-beat durations range from about 1% to about 100% based on the average inter-beat duration of the heart when no stimulation pulses are induced.

3. The method of claim 1, wherein the alternately shorter and longer inter-beat durations are generated on a randomized basis.

4. The method of claim 1, wherein the stimulation pulses are induced at a baseline frequency that increases the average contractile strength of the heart.

5. The method of claim 1, wherein the stimulation pulses are used to increase the intracellular calcium handling within the heart.

6. The method of claim 1, wherein the plurality of stimulation pulses alternate between fixed steady state frequency versus variable frequencies, and the average inter-beat duration remains constant.

7. A cardiac pacemaker device comprising:
pulse generator which emits a plurality of stimulation pulses, each of the plurality of stimulation pulses being separated by a plurality of inter-beat durations, respectively; and
a pacing algorithm configured to implement the pulse generator to generate alternately shorter and longer inter-beat durations independent of loading conditions and such that the average inter-beat duration remains constant.

8. The cardiac pacemaker device of claim 7, wherein the inter-beat durations range from about 1% to about 100% of the average inter-beat duration of the heart when no stimulation pulses are induced.

9. The cardiac pacemaker device of claim 7, wherein the alternately shorter and longer inter-beat durations are generated on a randomized basis.

10. The cardiac pacemaker device of claim 7, wherein the stimulation pulses are induced at a pattern that increases the average contractile strength of the heart without increasing average inter-beat duration.

11. The cardiac pacemaker device of claim 7, wherein the randomized stimulation pulses are induced at a constant average frequency that is less than about 4 Hz.

12. The cardiac pacemaker device of claim 7, wherein the randomized stimulation pulses are induced by independent changes in the magnitude of variation and the duration of the average inter-beat variation.

13. The cardiac pacemaker device of claim 7, wherein the stimulation pulse patterns increase the transport of calcium to the heart.

14. The cardiac pacemaker device of claim 7, wherein the plurality of stimulation pulses alternate between fixed steady state frequency versus variable frequencies, and the average inter-beat duration remains constant.

15. The cardiac pacemaker device of claim 7, further comprising;
a pulse actuator having a first state and a second state; and
wherein the pacing algorithm is configured to implement the pulse generator such that each of the plurality of stimulation pulses is separated by an inter-beat duration which is constant, and is equal to an average inter-beat duration, when the pulse actuator is in the first state; and
wherein the pacing algorithm is configured to implement the pulse generator to alternately shorten and lengthen the inter-beat duration, while maintaining the average inter-beat duration, when the pulse actuator is in the second state.
